(19) United States
(12) Patent Application Publication
(10) Pub. No.: US 2007/0293485 A1
(43) Pub. Date: Dec. 20, 2007

(54) DRUG ADMINISTRATION METHODS

(76) Inventors: John Lim, San Diego, CA (US); Jeffrey P. Whitten, Santee, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/757,273

(22) Filed: Jun. 1, 2007

Related U.S. Application Data
(60) Provisional application No. 60/803,864, filed on Jun. 3, 2006.
(60) Provisional application No. 60/805,975, filed on Jun. 27, 2006.
(60) Provisional application No. 60/821,115, filed on Aug. 1, 2006.
(60) Provisional application No. 60/857,988, filed on Nov. 8, 2006.
(60) Provisional application No. 60/939,326, filed on May 21, 2007.

(51) Int. Cl. A61K 31/5383

(52) U.S. Cl. 514/229.5

(57) ABSTRACT

Provided herein are methods for administering an efficacious amount of a compound of Formula I that reduce the severity of a potential adverse effect or obviate a potential adverse effect.

### CROSS-REFERENCE TO RELATED APPLICATIONS

This application claims priority under 35 U.S.C. § 119(e) to U.S. Provisional Application Ser. No. 60/803,864 filed Jun. 3, 2006; U.S. Provisional Application Ser. No. 60/805,975 filed Jun. 27, 2006; U.S. Provisional Application Ser. No. 60/821,115 filed Aug. 1, 2006; U.S. Provisional Application Ser. No. 60/857,988 filed Nov. 8, 2006; and U.S. Provisional Application Ser. No. 60/939,326 filed May 21, 2007. The contents of these documents are incorporated herein by reference in their entirety.

### FIELD OF THE INVENTION

The invention pertains to drug administration methods, and more specifically, to processes for effectively delivering an efficacious amount of a drug and concomitantly reducing or obviating potential adverse effects.

### DESCRIPTION

Drugs, such as anticancer drugs, can cause adverse side effects at therapeutic doses. Provided herein are methods for administering to a subject an anti-cancer drug in a manner that delivers an efficacious amount of the drug and reduces or obviates potential adverse drug effects. Thus, provided herein is a method for administering a compound to a subject, which comprises administering to the subject a pharmaceutical composition containing a compound of Formula I

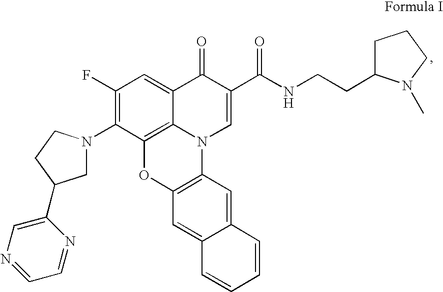

by infusion for a time period of over one hour in a day. A “compound” also may be a salt, ester, metabolite or prodrug of the compound of Formula I. A compound of Formula I sometimes is referred to as “Compound A” herein, as “CX-3543” and as “Quarfloxacin.” A subject often is human, and in certain embodiments, the subject sometimes is an animal, such as a dog, cat, rodent, ungulate, monkey, ape, bird, reptile or fish, for example.

In certain embodiments, the compound is at a dose of 160 mg/m² or greater, such as at a dose of 240 mg/m² or greater or 360 mg/m² or greater. The term “containing,” as used herein with respect to a pharmaceutical composition that contains the compound of Formula I, refers to the composition comprising the compound often with one or more other components (e.g., pharmaceutically acceptable carrier and/or excipient). The formulation of the pharmaceutical composition is suited to the method of administration, as known by persons of ordinary skill in the art. In certain embodiments, the compound is in a formulation comprising mannitol, phosphate buffer and polyethylene glycol (PEG) at a pH between about 5 to 8, such as for example 2% D-mannitol, 25 mM phosphate buffer, 10% PEG 300 and about 10% compound, where the formulation is at a pH of about 5.8. The latter formulation in certain embodiments sometimes comprises about 25 mM NaCl, which can result from the addition of an acid or base (e.g., HCl or NaOH) to adjust the pH of the formulation. Other components can be included in the formulation, and certain components can be removed or substituted with others, as can be determined by a person of ordinary skill in the art (e.g., U.S. Application Publication No. 20050085468, published on Apr. 21, 2005, entitled “Substituted quinobenzoxazine analogs”). In certain embodiments, the compound is administered in combination with another compound or procedure. Examples of procedures that may be used include but are not limited to radiotherapy and surgery. The compound may be administered in combination with a chemotherapeutic agent, and used to reduce cell proliferation, induce cell death, and/or ameliorate a cell proliferative disorder.

In some embodiments, the composition is administered for a time period of (a) greater than one hour; (b) over one hour to less than about six hours; (c) over one hour to less than about four hours; (d) about four hours; (e) about three hours; (f) about two hours; (g) about three hours to about five hours; (h) about five hours to about seven hours; (i) about six hours; (j) about 22 to about 26 hours or (k) about 24 hours. The composition in certain embodiments is administered once every day over a span of days (e.g., two, three, four, five, six or seven days), and sometimes the composition is administered on a cycle (e.g., a one-and-one-half, two, three or four week cycle). In certain embodiments, the composition is administered once a day for five consecutive days, on a three-week cycle (i.e., administering the composition once every day for five consecutive days, not administering the composition for two weeks, and then optionally repeating the cycle). In embodiments where the compound is administered for about five or more hours, administration can be periodic (e.g., once per week) and/or on a cycle (e.g., one administration per week for two weeks and then no administration for two weeks).

The term “about” as used herein refers to a value sometimes within 10% of the underlying parameter (i.e., plus or minus 10%), a value sometimes within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, an infusion time of “about two hours” includes a time period of 1.8 to 2.2 hours (i.e., 10% variation) or a time period of two hours (i.e., no variation) in certain embodiments.

In administration embodiments, the composition often is infused into the subject, frequently by intravenous infusion. Other types of infusion can be utilized, such as subcutaneous infusion, epidural infusion, arterial infusion and intraocular infusion, for example. Multiple types of apparatus for infusion administration are known, such as by use of implantable pumps and non-implantable portable or non-portable infusion pumps. Multiple types of infusion methodology also are known, including but not limited to continuous infusion, intermittent infusion or pulsatile infusion (e.g., U.S. Pat. No. 5,403,590). The infusion often is continuous during administration. The term “continuous” as used herein refers to a substantially uninterrupted administration. Certain infusion variables may fluctuate during the administration, such as, for example, the flow rate may fluctuate and may be pulsed. In some infusion embodiments, the flow rate may be reduced to substantially no flow for a period of time one or more times during the administration while the patient is connected to the infusion apparatus.

In certain administration embodiments, the severity of an adverse effect present during an infusion of one hour or less is reduced. The severity of an adverse effect can be ameliorated, eased, diminished or lessened, or obviated, abrogated or abolished by an administration method described herein. An adverse effect is any that may be caused by administration of a drug, including but not limited to, general disorders (e.g., port redness, fatigue, chills, chest tightness, fever), nutrition disorders (e.g., anorexia), nervous system disorders (e.g., involuntary movement, dysgeusia, headache, sensory neuropathy), fluid content disorders (e.g., elevated AST, proteinuria), cardiac disorders (e.g., hypertension), respiratory disorders (e.g., cough, throat tickle), skin disorders (e.g., alopecia), blood and lymph disorders (e.g., thrombocytopenia, anemia, leucopenia) and gastrointestinal conditions (e.g., diarrhea, nausea, vomiting, stomatitis). In certain embodiments, the adverse effect is a cough. Severity can be characterized according to a grading system (e.g., Grades 1 (mild), 2 (moderate) and 3 (severe)) known to persons of ordinary skill in the art.

Also featured is a method for administering a compound, which comprises administering a composition containing a compound of Formula I by continuous intravenous infusion for over one hour in a day, wherein the dose of the compound is 160 mg/m² or greater, whereby the severity of an adverse effect present during an infusion of one hour or less is reduced.

Provided also is a method for stabilizing or reducing the size of a tumor in a subject, which comprises administering to the subject a composition comprising a compound of Formula I by infusion for a time period of over one hour in a day. The tumor can be in any part of the subject, and in some embodiments, the tumor is selected from the group consisting of colon tumor, rectum tumor, prostate tumor, head tumor, neck tumor, neuroendocrine tumor, breast tumor, lung tumor, liver tumor, bone tumor and pancreatic tumor. The term “stabilizing” a tumor as used herein refers to the size of the tumor not substantially increasing after the drug is administered to the subject for a period of time (e.g., tumor size does not increase after one, two or three cycles of drug administration). Assessment of tumor size is known to the person of ordinary skill in the art (e.g., tomography, ultrasound, caliper methodologies).

Also featured is a method for administering a compound, which comprises administering to a subject a composition containing a compound of Formula I by infusion for a time period of about two hours to about six hours in a day, where the composition is administered once or twice per week at a dosage of 160 mg/m² or greater. In certain embodiments, the dosage is about 240 mg/m² or greater or about 360 mg/m² or greater. The composition may be administered for a time period of about two hours to about four hours.

Provided also is a method for stabilizing or reducing the size of a tumor in a subject, which comprises administering to the subject a composition comprising a compound of Formula I by infusion for a time period of about two hours to about six hours in a day, wherein the composition is administered once or twice per week at a dosage of 160 mg/m² or greater. In certain embodiments the dosage is about 240 mg/m² or greater or about 360 mg/m² or greater. The composition may be administered for a time period of about two hours to about four hours.

Also provided is a method for administering a compound to a subject, which comprises administering to the subject by infusion a composition containing (i) a compound of Formula I and (ii) a substance that reduces the severity of an adverse effect occurring when the compound is administered by infusion for a time period of less than one hour in a day. In some embodiments the adverse effect is a cough and the substance is selected from the group consisting of codeine, dextromethorphan, theobromine and chocolate. In certain embodiments, administration of the composition stabilizes or reduces the size of a tumor in the subject, where the tumor may be a colon tumor, rectum tumor, prostate tumor, head tumor, neck tumor, neuroendocrine tumor or pancreatic tumor.

Provided also herein is a method for administering a compound, which comprises administering a composition containing a compound of Formula I by intravenous infusion for about twenty-four hours, once in seven days, wherein the dose of the compound is about 160 mg/m² or greater. In some embodiments, the dose of the compound is about 360 mg/m² or greater; about 540 mg/m² or greater; about 720 mg/m² or greater; about 1053 mg/m² or greater; or about 1370 mg/m² or greater. The compound may be administered in one of more cycles, and a cycle may be three consecutive weeks in which the subject is administered the compound and one week in which the subject is not administered the compound, in certain embodiments. The infusion can be continuous infusion, and in some embodiments, the infusion is by a portable pump.

Also provided is a method for stabilizing or reducing the size of a tumor in a subject, which comprises administering to the subject a composition comprising a compound of Formula I by intravenous infusion for about twenty-four hours, once in a week, wherein the dose of the compound is about 160 mg/m² or greater. The tumor may be selected from the group consisting of colon tumor, rectum tumor, prostate tumor, head tumor, neck tumor, neuroendocrine tumor and pancreatic tumor in certain embodiments. The dose of the compound in some embodiments is about 360 mg/m² or greater; about 540 mg/m² or greater; about 720 mg/m² or greater; about 1053 mg/m² or greater; or about 1370 mg/m² or greater. The compound may be administered in one of more cycles, and a cycle may be three consecutive weeks in which the subject is administered the compound and one week in which the subject is not administered the compound, in certain embodiments. The infusion can be continuous infusion, and in some embodiments, the infusion is by a portable pump.

These and other embodiments are described hereafter in the Examples and in the Claims.

### BRIEF DESCRIPTION OF THE DRAWINGS

FIG. 1 shows the compound of Formula I exhibits linear pharmacokinetic behavior on the first day of dosing with proportional increases in AUC with dose level.

FIG. 2 shows characteristics of the group of patients enrolled in a study described in the Examples section.

FIG. 3 shows solid tumors treated in the patients in the group described in FIG. 2.

FIG. 4 shows average pharmacokinetic parameters for Compound A.

FIG. 5 shows evidence of biological activity of Compound A.

FIG. 6 shows an analysis of rat blood and plasma samples collected on Day 1 and Day 5 following five daily intravenous doses of Compound A.

FIG. 7 shows in vitro experiments in which human whole blood has been spiked with Compound A concentrations of 1, 5, 10, 25 and 50 μM.

FIGS. 8, 9 and 10 show the ability of fresh plasma to wash Compound A from blood cells after one, two and four washes.

FIGS. 11, 12 and 13 show analyses of Compound A in human blood from clinical trial subjects. Binding did not appear to be saturable when comparing Day 1 and Day 5 results (FIG. 11), and whole blood concentration of Compound A was approximately ten times that of plasma concentration on Day 1 (illustrated in FIG. 12) and on Day 5 (illustrated in FIG. 13).

### EXAMPLES

The examples set forth hereafter illustrate but do not limit the invention.

### Example 1

Administration of Compound A with One to Two Hour Infusion

The compound of Formula I (Compound A), referred to herein as Compound A, is a novel small molecule designed to target a protein-rDNA interaction that is critical to cancer cells and thus induces apoptosis. Preclinically, Compound A demonstrated potency in suppressing xenograft tumor growth with a broad therapeutic window, and no drug resistance has been observed in vitro. The rate of ribosomal RNA (rRNA) biosynthesis defines the proliferative state of cells, and this process is highly deregulated and increased in cancer cells. Indirect inhibition of rRNA biosynthesis through the targeting of upstream kinase pathways has been demonstrated with drugs such as bevacizumab, trastuzumab, imatinib and sunitinib. In contrast, Compound A directly inhibits aberrant rRNA biogenesis in cancer cells by disrupting an essential protein-rDNA quadruplex interaction over-expressed in cancer cells (e.g., U.S. Patent Application No. 60/775,924 filed on Feb. 22, 2006 and other patent applications that incorporate it by reference). Derived from the structural template of the fluoroquinolone class of drugs, Compound A rapidly induces selective apoptosis in malignant cells in vitro and tumor growth inhibition in in vivo xenograft models.

The objectives of the study described hereafter are to determine the maximum tolerated dose (MTD) and dose limiting toxicities (DLTs), to establish the pharmacokinetics (PKs), and to determine the recommended dose for further clinical development of Compound A.

Methods

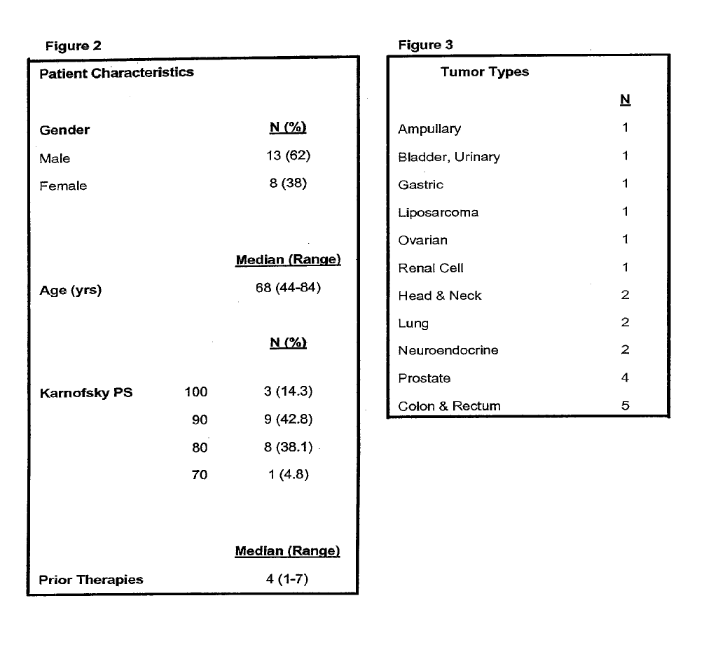

Eligible patients with advanced solid tumors or lymphomas whose tumors had progressed on, or for whom there are no standard therapies, receive Compound A in successive dose cohorts at: 10, 20, 40, 80 and 160 mg/m². Dosing is by one or two hour intravenous infusion daily for five consecutive days repeated on a three week cycle. Therapy is continued until the patient shows signs of intolerance to Compound A, or evidence of advancing disease. Response by RECIST is determined after every 2 cycles. FIG. 2 shows characteristics of the group of patients enrolled in the study, and FIG. 3 shows the solid tumors treated in the patients.

Results

Twenty-one patients with solid tumors (3-8 patients per cohort) have received intravenous Compound A, and doses have been well tolerated; nine grade 3 adverse events have been reported during the study, but none of these are deemed related to Compound A. No objective tumor responses have been observed, but three patients have had disease stabilization durations of longer than four months. Compound A has demonstrated linearity in PK parameters between the dose cohorts, with a terminal half life of approximately 10 hours following the first dose.

No drug-related serious adverse events have been observed during the study. Adverse events (AEs) deemed at least possibly related to drug have been reported at all studied dose levels, but all have been grade 1 or 2 in severity. Some patients experienced a transient grade 1 cough with a one-hour infusion at the highest dose level (160 mg/m²) that resolved spontaneously upon completion of the infusion. When the protocol was amended to extend the infusion duration to two hours, the cough resolved. Generally Compound A has been very well tolerated, with no observations to date of dose limiting toxicities. Since the maximum tolerated dose (MTD) has not yet been defined with this highest dose, the protocol has been amended to allow for further dose escalations to levels above 160 mg/m².

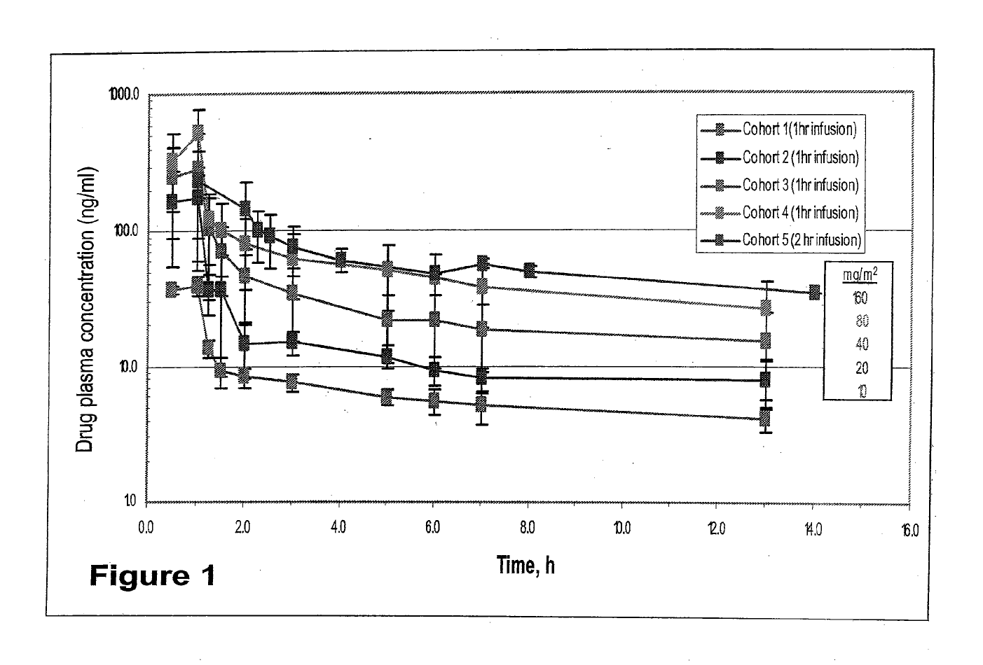

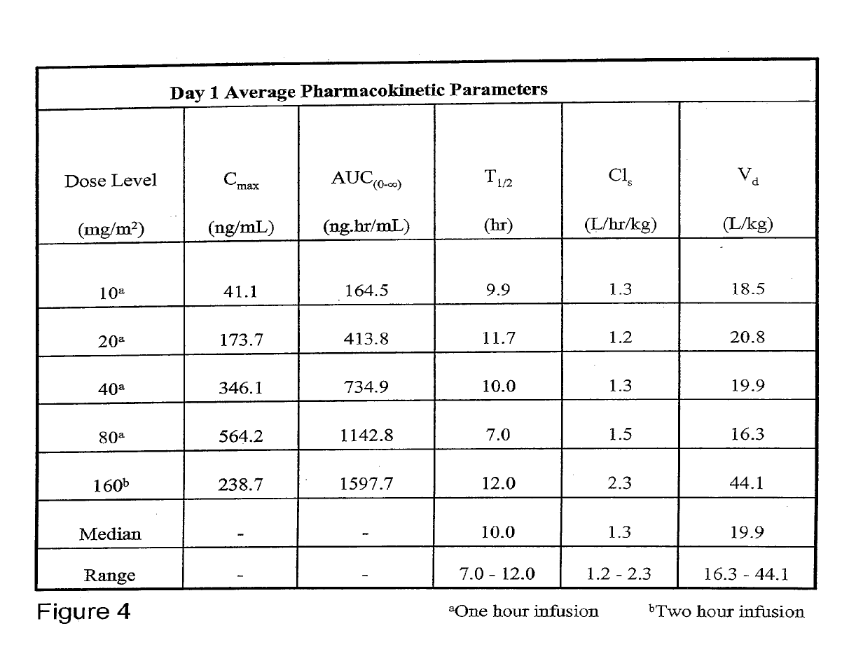

Compound A exhibits linear pharmacokinetic behavior on day 1 of dosing, with proportional increases in AUC with dose level (FIG. 1). Plasma half life remained consistent at approximately 10 hours on day 1 across all the dose levels (FIG. 4). Extending the infusion duration to 2 hours at the 160 mg/m² dose level decreased the maximum plasma concentration (Cₘₐₓ) as expected, but AUC remained linear, increasing in proportion with the dose level.

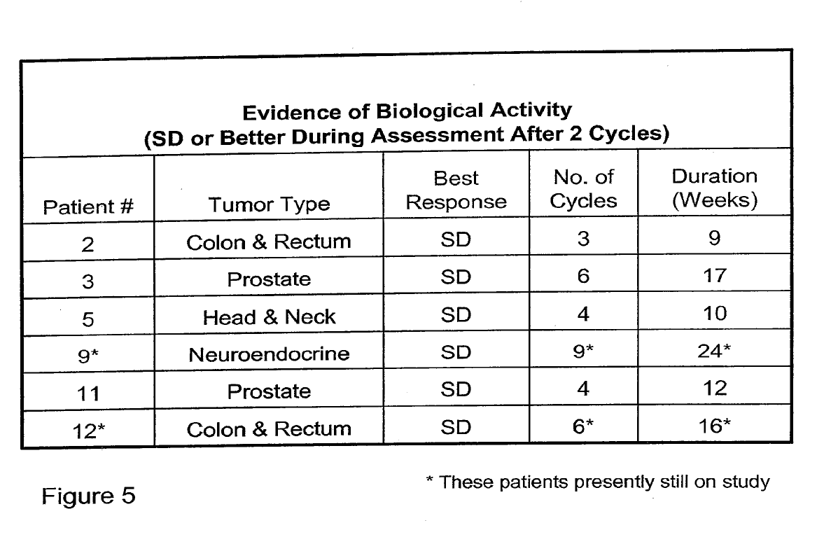

Six patients presented stable disease (SD) at the disease evaluation following two cycles of treatment, and three of these had disease stabilization for at least 4 months. Median duration of disease stabilization for these patients is 14 weeks, which is within a range 9 to 24 weeks (FIG. 5).

Conclusions

Compound A has shown no drug related toxicity and has predictable PKs. No DLTs have yet been observed at the highest protocol dose level, and the MTD remains to be defined in this phase I study. Further patient enrollment with an expanded dose escalation is ongoing. Compound A is well tolerated, with no reports of serious adverse events deemed related to drug. Reported adverse experiences to date have been graded mild to moderate in severity. A transient grade 1 cough was noted with the one hour infusion at the highest dose level of 160 mg/m². This cough resolves spontaneously upon the completion of infusion, and has not limited dose administration when the infusion duration is extended to 2 hours. The maximum tolerated dose (MTD) has not yet been defined, and the protocol has been amended to allow continued dose escalations to levels above 160 mg/m². Stable Disease (SD) has been observed in six patients when assessed after 2 cycles, with the longest period of stable disease to date of 24 weeks. Day 1 pharmacokinetic parameters are linear and predictable at all dose levels studied.

### Example 2

### Administration of Compound A with Six Hour Infusion

Compound A is designed to inhibit over-expressed ribosomal RNA synthesis in cancer cells by disrupting an essential protein-rDNA quadruplex complex thereby inducing selective apoptosis. A Phase I clinical trial for Compound A has been undertaken to determine the dose limiting toxicities (DLTs), maximum tolerated dose (MTD) and pharmacokinetics (PK) of this agent with administration by infusion for about six hours per day.

Methods

Eligible patients with advanced solid tumors received Compound A in successive dose cohorts at: 10, 20, 40, 80, 160, 240, 360 and 480 mg/m². Drug is administered by daily intravenous infusion on the first five consecutive days of a three week cycle and the infusion duration has varied from one hour to six hours. Response by RECIST is determined after every 2 cycles.

Results

Forty-two patients (M/F:25/17; median age 69, range 44-83) with colorectal cancer (9), prostate cancer (7), lung cancer (5), pancreatic cancer (5), head and neck cancer (3), renal cancer (2) and others (11) were treated with intravenous Compound A for a median of 2 cycles (range: 1-26). Patient attributes are described in greater detail in the following Table 1.

TABLE 1
Patient Characteristics

| Age-median (range) | 69 (44-83) |
|---|---|
| Gender | |
| Male | 25 |
| Female | 17 |
| Karnofsky Performance Status | |
| 100% | 10 |
| 90% | 19 |
| 80% | 12 |
| 70% | 1 |
| Tumor Types | |
| Colorectal | 9 |
| Prostate | 7 |
| Lung | 5 |
| Pancreas | 5 |
| Head and Neck | 3 |
| Renal | 2 |
| Other | 11 |
| Prior therapies-median (range) | 3 (1-7) |

One patient has remained on-study for over one and a half years, and presently continues on treatment. The duration of treatment for study patients is presented in the following Table 2.

TABLE 2
Duration of Treatment for Study Patients

| Compound A | Total | Total | Range in | Number |
|---|---|---|---|---|
| (CX-3543) | Number of | Number of | Number of | of |
| Dose Level | Patients | Cycles in | Cycles | Patients |
| (mg/m²) | in Cohort | the Cohort | Administered | with DLT |
| 10 | 3 | 11 | 2-6 | 0 |
| 20 | 4 | 8 | 1-3 | 0 |
| 40 | 3 | 32 | 2-26^{a} | 0 |
| 80 | 3 | 15 | 1-12^{b} | 0 |
| 160 | 8 | 16 | 1-6 | 0 |
| 240 | 3 | 6 | 2 | 0 |
| 360^{c} | 14 | 27 | 1-2 | 1 |
| 480 | 4 | 6 | 1-2 | 2 |

^{a}Including one patient who was internally dose escalated to 240 mg/m². This patient remains on study at 240 mg/m².
^{b}Including one patient who was internally dose escalated to 160 mg/m².
^{c}This dose level was defined as the MTD.

No drug-related serious adverse events (SAEs) have been encountered. DLTs of infusion-related cough, dyspnea without a decrease in O₂ saturation, muscle cramps and headache were identified at 480 mg/m². One patient receiving 360 mg/m² was discontinued due to transient, infusion-associated hypertension. These toxicities were fully reversible upon slowing or interruption of the infusion. MTD was determined to be 360 mg/m². Other drug-related adverse events were of mild to moderate intensity. A listing of adverse events observed in at least two patients, and that are deemed at least possibly related to Compound A, is presented in the following Table 3.

Four patients have had stable disease for longer than four months and one patient, who has experienced disease stabilization for longer than one year, is currently continuing on study. In particular, (a) at the 10 mg/m² dose level, one patient with prostate cancer had stable disease for six cycles; (b) at the 160 mg/m² dose level, one patient with liposarcoma had stable disease for six cycles; (c) at the 160 mg/m² dose level, one patient with anorectal cancer (internal dose escalation from the 80 mg/m2 dose level) had stable disease for twelve cycles; and (d) at the 240 mg/m² dose level, one patient with neuroendocrine islet cell cancer of the pancreas (internal dose escalation from the 40 mg/m² dose level) remains on-study after completing 26 cycles of Compound A.

Compound A demonstrated an increasing plasma terminal half life at the higher dose levels and this has now been characterized as due to a “reservoir” effect from Compound A reversibly binding and then gradually being released from blood cells. Specifically, the plasma terminal half life of Compound A was observed to increase with repeated dosing and with each dose level escalation, but without significant accumulation in Compound A plasma concentration. The pharmacokinetic sampling procedure was amended to collect whole blood and plasma specimens for analysis at each time point, and Compound A was observed to bind reversibly to blood cells. Analysis has determined that Compound A whole blood concentration is nearly ten times that of plasma concentration. The reversible binding and subsequent slow release of Compound A from blood cells provides a “reservoir” effect to extend the Compound A plasma terminal half life. Further details of these observations are described in an Example that follows. This pharmacokinetic property allows consideration of a less frequent dosing schedule, and a phase I study of weekly intravenous Compound A administration is being implemented.

Conclusions

Compound A administered as a six hour infusion for five consecutive days of a three week cycle is well tolerated and has shown disease stabilization in a number of patients enrolled in a Phase I study. The MTD for Compound A has been identified as 360 mg/m², with reversible cough, dyspnea and headache as DLTs at the stopping dose of 480 mg/m². Compound A is well tolerated with no drug related SAEs, and other reported adverse events have been mild to moderate in intensity. Four patients have shown durable disease stabilization of 4 months or longer, and one of these patients continues on-study after more than one and a half years. Pharmacokinetic analysis has shown Compound A to have an extended plasma terminal half life due to reversible blood cell binding. This allows consideration of a less frequent dosing schedule, and a phase I study of weekly intravenous Compound A administration is being implemented.

TABLE 3
Adverse Events Deemed at Least Possibly Related to Compound A

| | 10-80 mg/m² | | 160 mg/m² | | 240 mg/m² | | 360 mg/m² | | 480 mg/m² | |
|---|---|---|---|---|---|---|---|---|---|---|
| Dose Level | N = 13 | | N = 8 | | N = 3 | | N = 14 | | N = 4 | |
| # Pts. enrolled | Grades | Grade | Grades | Grade | Grades | Grade | Grades | Grade | Grades | Grade |
| Adverse Event^{a} | 1/2 | 3 | 1/2 | 3 | 1/2 | 3 | 1/2 | 3 | 1/2 | 3 |
| Anemia | 2 | — | — | 1 | 1 | — | 2 | — | 1 | — |
| Thrombocytopenia | 1 | — | — | — | — | — | 1 | — | — | 1 |
| Diarrhea | 2 | — | 2 | — | — | — | 1 | — | 1 | — |
| Nausea | 2 | — | 2 | — | 1 | — | 5 | — | — | — |
| Stomatitis | 1 | — | 1 | — | — | — | — | — | — | — |
| Vomiting | 1 | — | 1 | — | 1 | — | 2 | — | 1 | — |
| Chest discomfort | — | — | 2 | — | 1 | — | 1 | — | — | — |
| Chest pain | — | — | 1 | — | 1 | — | — | — | — | — |
| Chills | 1 | — | 1 | — | — | — | — | — | — | — |
| Fatigue | 4 | 1 | 1 | — | 1 | — | 7 | — | — | 1 |
| Fever | 2 | — | 2 | — | — | — | 1 | — | 1 | — |
| Increased creatinine | — | — | — | — | — | — | 1 | — | 1 | — |
| Anorexia | 5 | — | — | — | 1 | — | 1 | — | 1 | — |
| Muscle spasms | — | — | — | — | 1 | — | — | 1^{b} | 1 | 1 |
| Extremity pain | — | — | — | — | — | — | 2 | 1^{b} | — | — |
| Dysgeusia | 1 | — | — | — | 1 | — | — | — | — | — |
| Dyskinesia | 1 | — | 1 | — | — | — | — | — | — | — |
| Headache | 1 | — | 1 | — | — | — | 5 | 1^{b} | 1 | 1 |
| Cough | 1 | — | 5 | — | 3 | — | 9 | — | 2 | 1 |
| Dyspnea | — | — | — | — | 1 | — | — | — | 1 | 1 |
| Throat irritation | — | — | 2 | — | — | — | — | — | — | — |
| Hypertension | — | — | 1 | — | — | — | 3^{b} | — | 1 | — |

No grade 4 adverse events were reported.
^{a}Listed adverse events have been reported in two or more patients taken across all dose levels.
^{b}Adverse events reported by one patient who was dose reduced from 480 mg/m².

### Example 3

### Compound A Interacts with Blood Cells

Pharmacokinetic analyses of plasma samples collected on Day 1 and Day 5 of dosing for every patient were performed in cohort batches during the conduct of the phase I study of Compound A in refractory solid tumors and lymphomas (Protocol C3-05-001). These analyses revealed that plasma terminal half life of Compound A appeared to be extended on Day 5 when compared with Day 1, and half life also had the trend of being prolonged with each dose level escalation. Saturation of elimination was an unlikely cause of the extended half life because there was no apparent accumulation of drug in plasma. Moreover, while it is known that Compound A is eliminated unchanged in urine, the urinary clearance of drug calculated from a 12-hour quantitative urine collection revealed a clearance rate at 10% of the glomerular filtration rate.

These observations support blood cell binding by Compound A as the reason for the extended plasma terminal half life. To investigate this possibility, blood and plasma samples were collected from rats on Day 1 and Day 5, and analyzed following five daily intravenous doses of Compound A. The result of this analysis is illustrated in FIG. 6. This study revealed that the concentration of Compound A attained in whole blood was approximately ten times that attained in plasma on both Days 1 and 5, confirming binding to blood cells.

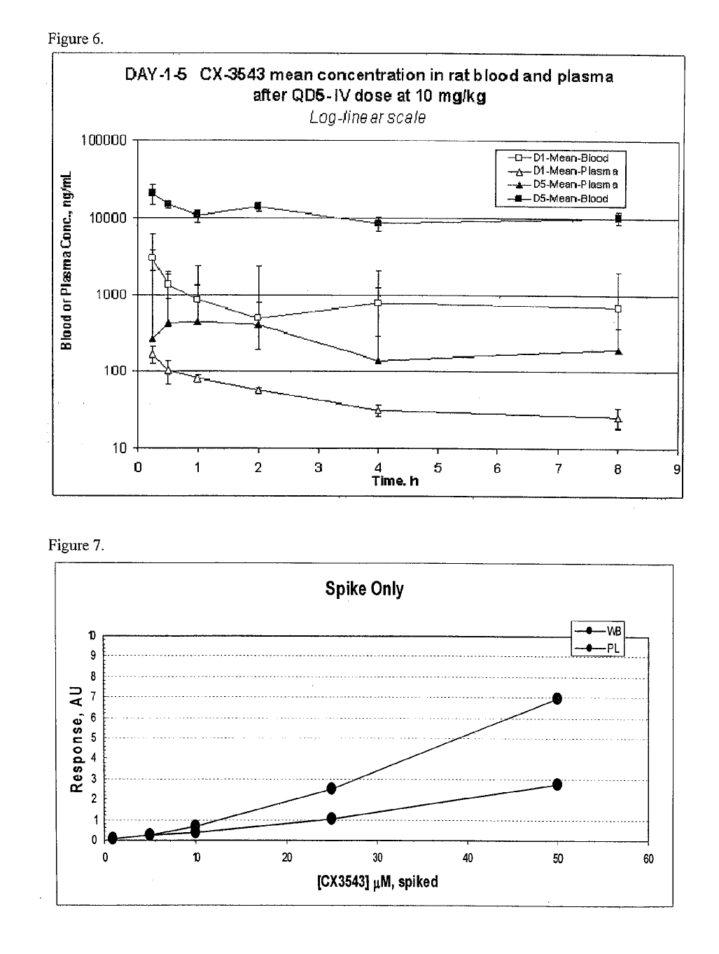

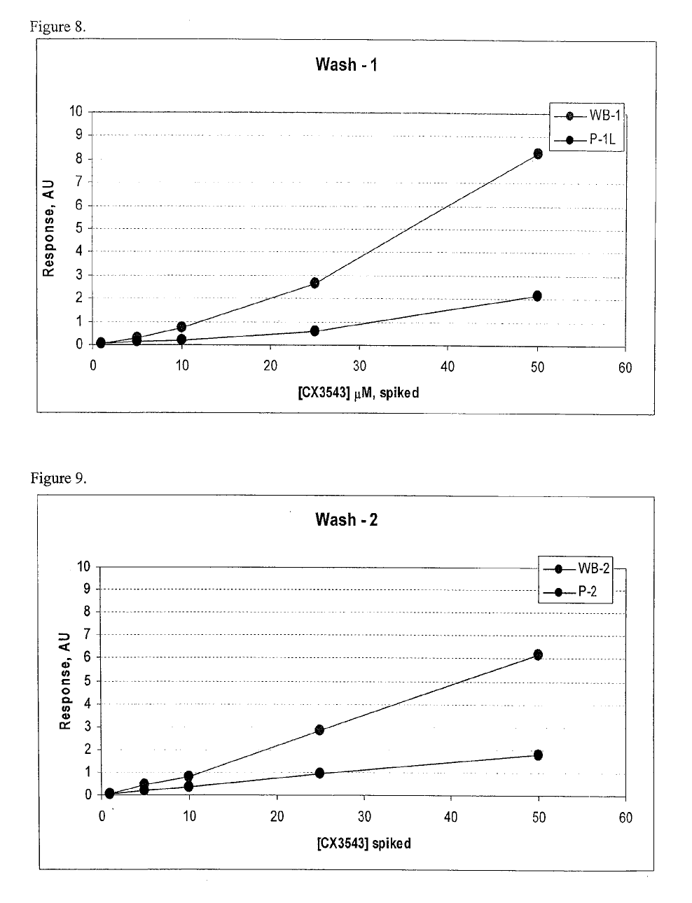

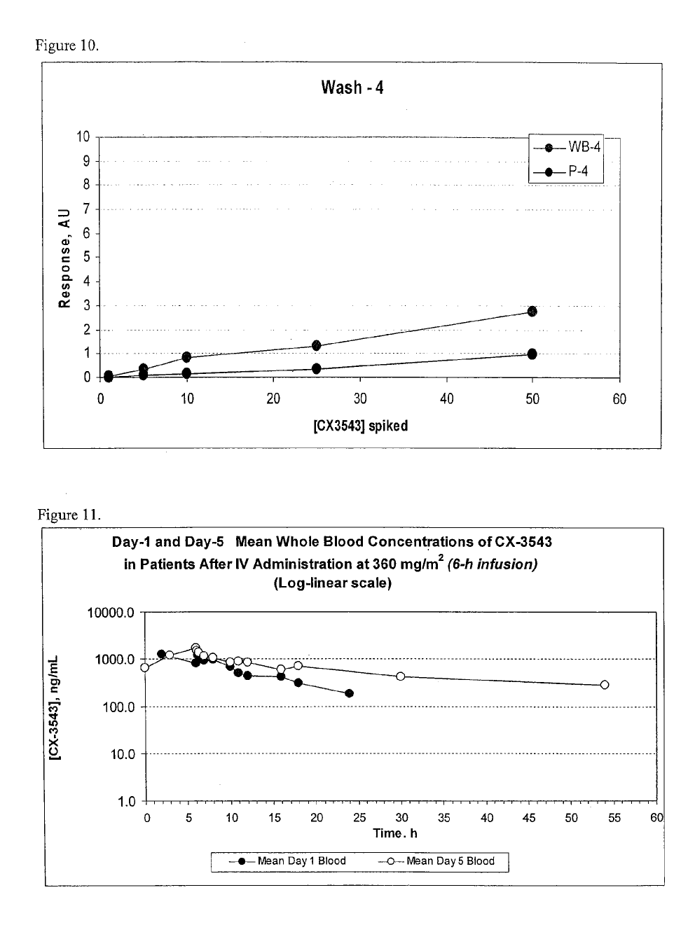

To test if this binding also occurs with human blood cells, an in vitro experiment was conducted with human whole blood spiked with Compound A concentrations of 1, 5, 10, and 50 μM. This analysis revealed a concentration dependent binding of Compound A to human blood cells which was not saturable, even at 50 μM. Results of the analysis of whole blood and plasma are illustrated in FIG. 7. The permanence of the binding was studied by attempting to “wash off” bound drug with fresh plasma. This study showed that binding is easily reversible, with drug in plasma being replaced from the “reservoir” of drug bound to blood cells. This reversible binding to blood cells is the reason for the observed extended plasma terminal half life. The ability of fresh plasma to wash off Compound A from blood cells after one, two and four washes is illustrated in FIGS. 8, 9, and 10, respectively.

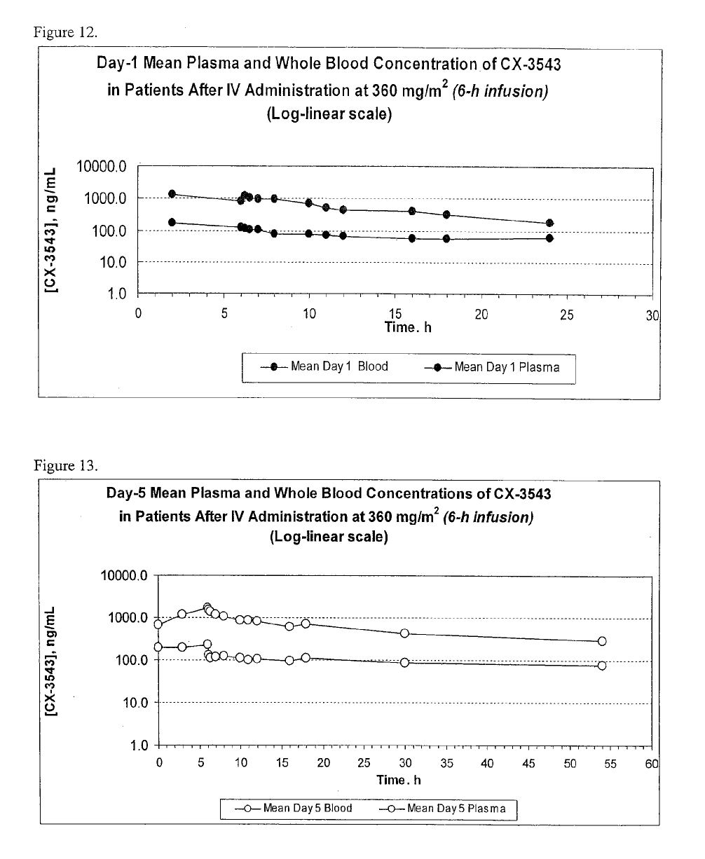

The pharmacokinetic sampling procedure in the phase I clinical trial was amended to allow for whole blood and plasma to be collected from study patients. When these samples were analyzed, the binding of Compound A to blood cells was again evident. This binding did not appear to be saturable when comparing Day 1 and Day 5 results (illustrated in FIG. 11), and once again, whole blood concentration of Compound A was approximately ten times that of plasma concentration on Day 1 (illustrated in FIG. 12) and on Day 5 (illustrated in FIG. 13).

### Example 4

### Once a Week Administration of Compound A

Patients will receive their assigned dose of study drug administered as a 24 hour intravenous infusion once weekly for 3 weeks followed by one week without therapy. Patients who successfully complete a 4-week (28 day) treatment cycle without evidence of significant treatment-related toxicity or progressive disease will continue to receive treatment.

When used in the clinic, the appropriate dose for the patient is diluted with an equal amount of aqueous dextrose 5% solution in water for injection rounded to the nearest 25 mL before administration via intravenous infusion over 24 hours. The initial 24-hour administration of study drug (Cycle 1, Day 1) will occur in the clinic with capabilities for overnight patient observation and PK sample collection. In-patient admission (<24 hours) or provision of an overnight staffed research center will be necessary for observation and specimen collection during the first administration of study drug. Portable infusion pumps will be used for out-patient administration of study drug subsequent to Cycle 1 Day 1 treatment. Ancillary support for the maintenance of the portable infusion pumps and at-home infusion may be necessary. All infusions of study drug will be administered by medically qualified site staff under the supervision of an Investigator at an Institution listed on the Form FDA 1572.

The dose escalation steps in this study will be based on the observation of clinical toxicity. To minimize the number of patients who may be treated at sub-therapeutic levels, the study design will include a single patient dose escalation period for the first 3 lowest dose levels (i.e., at 360, 540 and 810 mg/m²). If the patient does not experience Grade ≧2 toxicity (according to NCI-CTCAE version 3.0) during the single patient dose escalation period, then the following patient will be treated at the next higher dose level of the dose escalation scheme. If, however, the patient experiences Grade ≧2 toxicity during this period, then the cohort will be expanded to include 3 patients, and all subsequent dose escalation will be in 30% increments following the standard 3+3 dose escalation design until the MTD is determined.

An estimated dose escalation scheme following a Level 1: Level 2: Level 3: Level 4: Level 5 approach will be implemented with the following levels: 360 mg/m² once weekly×3; one week of rest 540 mg/m² once weekly×3; one week of rest 720 mg/m² once weekly×3; one week of rest 1053 mg/m² once weekly×3; one week of rest 1370 mg/m² once weekly×3; one week of rest. If the stopping dose is not achieved at the 1370 mg/m² cohort, then dose escalations may continue in 30% dose increments. During the single patient dose escalation period, if the patient does not develop CTCAE Grade 2 or greater drug-related toxicity after Day 15 on-study, then the next patient may be enrolled at the next higher dose level. During the 3+3 dose escalation portion, the first patient enrolled at that dose level must complete Day 15 with no observed DLTs before a second patient and a third patient is enrolled to receive treatment in that cohort one week apart. If no patients in the cohort experience a Dose Limiting Toxicity (DLT) after treatment Day 15 for the final patient in the cohort, then a dose escalation to the next higher cohort may proceed.

If a DLT is observed in 1 out of 3 patients at a given dose level, then up to 3 additional patients will be enrolled (i.e., for a total of six patients) and treated at that dose level. If only 1 of 6 patients in any expanded cohort has a DLT, then the dose will be escalated to the next higher dose level. If 2 patients out of the 6 at that dose level have DLTs, then the dose will be decreased to the previous dose level and up to 3 additional patients may be enrolled if that lower dose level has fewer than six patients. At the discretion of the Investigators and Sponsor, the escalated dose selected may be less than the next higher dose level in the sequence. If 2 patients out of the six at that dose level have DLTs, then the dose will be decreased to the previous dose level and up to 3 additional patients may be enrolled if that lower dose level has fewer than 6 patients. The Maximum Tolerated Dose (MTD) is defined as the highest level where fewer than 2 of 6 patients have DLTs. Once an MTD has been established, up to 10 additional patients will be enrolled at the MTD level for confirmation of safety, pharmacokinetics and to evaluate additional pharmacodynamic parameters. Patients that have experienced a DLT at any dose level will not receive additional doses at that level, but may be offered the option to continue doses at the next lower level if this is considered safe by the Investigator and the Sponsor.

If deemed safe and appropriate, the dose may be escalated within a patient up to the dose level immediately below the current highest safe dose level or to the MTD, provided that the patient has tolerated at least four cycles of test drug at his/her assigned dose level. If this higher dose is subsequently deemed intolerable, then the patient may be offered the option to resume doses at his/her previously assigned dose level.

### Example 5

### Examples of Embodiments

Provided hereafter are non-limiting examples of embodiments of the invention.

1. A method for administering a compound to a subject, which comprises administering to the subject a composition containing a compound of Formula I

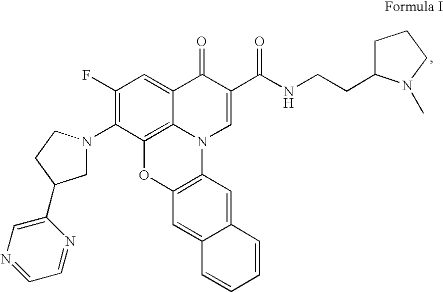

by infusion for a time period of over one hour in a day.

2. The method of aspect 1, wherein the compound is at a dose of 160 mg/m² or greater.

3. The method of aspect 1, wherein the composition is administered for a time period of over one hour.

4. The method of aspect 1, wherein the composition is administered for a time period of over one hour and less than about six hours.

5. The method of aspect 1, wherein the composition is administered for a time period of over one hour and less than about four hours.

6. The method of aspect 1, wherein the composition is administered for a time period of about two hours.

7. The method of aspect 1, wherein the composition is administered for a time period of two hours.

8. The method of aspect 1, wherein the composition is administered for a time period of about five to about seven hours.

9. The method of aspect 1, wherein the composition is administered for a time period of about six hours.

10. The method of aspect 1, wherein the composition is administered for a time period of about three hours to about five hours.

11. The method of aspect 1, wherein the composition is administered for a time period of about four hours.

12. The method of aspect 1, wherein the composition is administered for a time period of about 22 to about 26 hours.

13. The method of aspect 1, wherein the composition is administered for a time period of about 24 hours.

14. The method of aspect 1, wherein the composition is administered once every day for five days.

15. The method of aspect 14, wherein the composition is administered on a three-week cycle.

16. The method of any one of the preceding aspects, whereby the severity of an adverse effect present during an infusion of one hour or less is reduced.

17. The method of aspect 16, wherein the adverse effect is a cough.

18. The method of aspect 1, wherein the infusion is intravenous infusion.

19. A method for administering a compound, which comprises administering a composition containing a compound of Formula I by continuous intravenous infusion for over one hour in a day, wherein the dose of the compound is 160 mg/m² or greater, whereby the severity of an adverse effect present during an infusion of one hour or less is reduced.

20. A method for stabilizing or reducing the size of a tumor in a subject, which comprises administering to the subject a composition comprising a compound of Formula I by infusion for a time period of over one hour in a day.

21. The method of aspect 20, wherein the tumor is selected from the group consisting of colon tumor, rectum tumor, prostate tumor, head tumor, neck tumor, neuroendocrine tumor and pancreatic tumor.

22. A method for administering a compound, which comprises administering to a subject a composition containing a compound of Formula I by infusion for a time period of about two hours to about six hours in a day, wherein the composition is administered once or twice per week at a dosage of 160 mg/m² or greater.

23. The method of aspect 22, wherein the dosage is about 240 mg/m² or greater.

24. The method of aspect 22, wherein the dosage is about 360 mg/m² or greater.

25. The method of aspect 22, wherein the composition is administered for a time period of about two hours to about four hours.

26. The method of aspect 22, wherein the composition is administered once per week.

27. The method of aspect 22, wherein the composition is administered twice per week.

28. A method for stabilizing or reducing the size of a tumor in a subject, which comprises administering to the subject a composition comprising a compound of Formula I by infusion for a time period of about two hours to about six hours in a day, wherein the composition is administered once or twice per week at a dosage of 160 mg/m² or greater.

29. The method of aspect 28, wherein the dosage is about 240 mg/m² or greater.

30. The method of aspect 28, wherein the dosage is about 360 mg/m² or greater.

31. The method of aspect 28, wherein the composition is administered for a time period of about two hours to about four hours.

32. The method of aspect 28, wherein the tumor is selected from the group consisting of colon tumor, rectum tumor, prostate tumor, head tumor, neck tumor, neuroendocrine tumor and pancreatic tumor.

33. The method of aspect 28, wherein the composition is administered once per week.

34. The method of aspect 28, wherein the composition is administered twice per week.

35. A method for administering a compound to a subject, which comprises administering to the subject by infusion a composition containing (i) a compound of Formula I and (ii) a substance that reduces the severity of an adverse effect that occurs when the compound is administered by infusion for a time period of less than one hour in a day.

36. The method of aspect 35, wherein the infusion is for a time period of over one hour in a day.

37. The method of aspect 35, wherein the compound of Formula I is at a dose of 160 mg/m² or greater.

38. The method of aspect 35, wherein the dosage of the compound of Formula I is about 240 mg/m² or greater.

39. The method of aspect 35, wherein the dosage of the compound of Formula I is about 360 mg/m² or greater.

40. The method of aspect 35, wherein the composition is administered for a time period of over one hour and less than about six hours.

41. The method of aspect 35, wherein the composition is administered for a time period of about 22 hours to about 26 hours.

42. The method of aspect 35, wherein the composition is administered for a time period of about 24 hours.

43. The method of aspect 35, wherein the composition is administered once every day for five days.

44. The method of aspect 43, wherein the composition is administered on a three-week cycle.

45. The method of aspect 35, wherein the adverse effect is a cough.

46. The method of aspect 45, wherein the substance is selected from the group consisting of codeine, dextromethorphan, theobromine and chocolate.

47. The method of aspect 45, wherein the substance is codeine.

48. The method of aspect 35, wherein administration of the composition stabilizes or reduces the size of a tumor in the subject.

49. The method of aspect 48, wherein the tumor is selected from the group consisting of colon tumor, rectum tumor, prostate tumor, head tumor, neck tumor, neuroendocrine tumor and pancreatic tumor.

50. The method of aspect 35, wherein the infusion is intravenous infusion.

51. The method of any one of the preceding aspects, wherein the infusion is continuous infusion.

52. A method for administering a compound, which comprises administering a composition containing a compound of Formula I by intravenous infusion for about twenty-four hours, once in seven days, wherein the dose of the compound is about 160 mg/m² or greater.

53. The method of embodiment 52, wherein the dose of the compound is about 360 mg/m² or greater.

54. The method of embodiment 52, wherein the dose of the compound is about 540 mg/m² or greater.

55. The method of embodiment 52, wherein the dose of the compound is about 720 mg/m² or greater.

56. The method of embodiment 52, wherein the dose of the compound is about 1053 mg/m² or greater.

57. The method of embodiment 52, wherein the dose of the compound is about 1370 mg/m² or greater.

58. The method of any one of embodiments 52-57, wherein the compound is administered in one of more cycles.

59. The method of embodiment 58, wherein a cycle is three consecutive weeks in which the subject is administered the compound and one week in which the subject is not administered the compound.

60. The method of any one of embodiments 52-59, wherein the infusion is continuous infusion.

61. The method of any one of embodiments 52-60, wherein the infusion is by a portable pump.

62. A method for stabilizing or reducing the size of a tumor in a subject, which comprises administering to the subject a composition comprising a compound of Formula I by intravenous infusion for about twenty-four hours, once in a week, wherein the dose of the compound is about 160 mg/m² or greater.

63. The method of embodiment 62, wherein the tumor is selected from the group consisting of colon tumor, rectum tumor, prostate tumor, head tumor, neck tumor, neuroendocrine tumor and pancreatic tumor.

64. The method of embodiment 62, wherein the dose of the compound is about 360 mg/m² or greater.

65. The method of embodiment 62, wherein the dose of the compound is about 540 mg/m² or greater.

66. The method of embodiment 62, wherein the dose of the compound is about 720 mg/m² or greater.

67. The method of embodiment 62, wherein the dose of the compound is about 1053 mg/m² or greater.

68. The method of embodiment 62, wherein the dose of the compound is about 1370 mg/m² or greater.

69. The method of any one of embodiments 62-68, wherein the compound is administered in one of more cycles.

70. The method of embodiment 69, wherein a cycle is three consecutive weeks in which the subject is administered the compound and one week in which the subject is not administered the compound.

71. The method of any one of embodiments 62-70, wherein the infusion is continuous infusion.

72. The method of any one of embodiments 62-71, wherein the infusion is by a portable pump.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claim.

What is claimed is:

1. A method for administering a compound to a subject, which comprises administering to the subject a composition containing a compound of Formula I 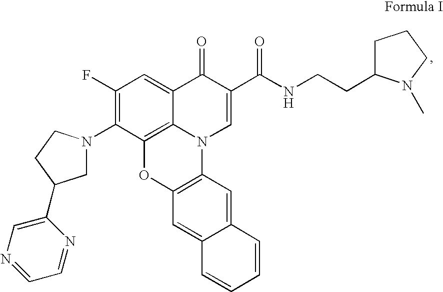
by infusion for a time period of over one hour in a day.

2. The method of claim 1, wherein the compound is at a dose of 160 mg/m² or greater.

3. The method of claim 1, wherein the composition is administered for a time period of over one hour and less than about six hours.

4. The method of claim 1, wherein the composition is administered for a time period of about five to about seven hours.

5. The method of claim 1, wherein the composition is administered for a time period of about three hours to about five hours.

6. The method of claim 1, wherein the composition is administered for a time period of about 22 to about 26 hours.

7. The method of claim 1, wherein the composition is administered once every day for five days.

8. The method of claim 1, whereby the severity of an adverse effect present during an infusion of one hour or less is reduced.

9. A method for stabilizing or reducing the size of a tumor in a subject, which comprises administering to the subject a composition comprising a compound of Formula I: 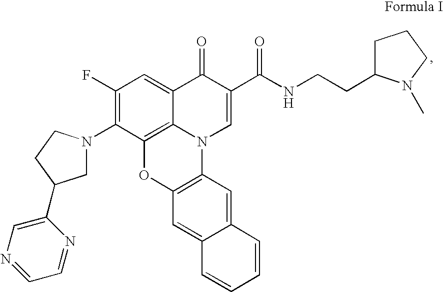
by infusion for a time period of over one hour in a day.

10. The method of claim 9, wherein the tumor is selected from the group consisting of colon tumor, rectum tumor, prostate tumor, head tumor, neck tumor, neuroendocrine tumor and pancreatic tumor.

11. The method of claim 9, wherein the dosage is about 240 mg/m² or greater.

12. The method of claim 9, wherein the dosage is about 360 mg/m² or greater.

13. A method for administering a compound to a subject, which comprises administering to the subject by infusion a composition containing (i) a compound of Formula I: 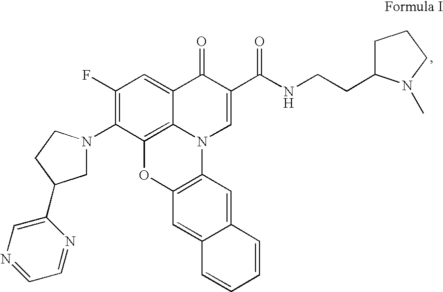
and (ii) a substance that reduces the severity of an adverse effect that occurs when the compound is administered by infusion for a time period of less than one hour in a day.

14. The method of claim 13, wherein the infusion is for a time period of over one hour in a day.

15. The method of claim 13, wherein the compound of Formula I is at a dose of 160 mg/m² or greater.

16. The method of claim 13, wherein the dosage of the compound of Formula I is about 360 mg/m² or greater.

17. The method of claim 13, wherein the composition is administered for a time period of over one hour and less than about six hours.

18. The method of claim 13, wherein the composition is administered for a time period of about 22 hours to about 26 hours.

19. The method of claim 18, wherein the substance is selected from the group consisting of codeine, dextromethorphan, theobromine and chocolate.

20. The method of claim 13, wherein administration of the composition stabilizes or reduces the size of a tumor in the subject.

21. The method of claim 20, wherein the tumor is selected from the group consisting of colon tumor, rectum tumor, prostate tumor, head tumor, neck tumor, neuroendocrine tumor and pancreatic tumor.

22. A method for administering a compound, which comprises administering a composition containing a compound of Formula I: 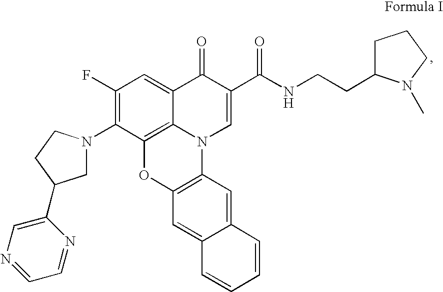
by intravenous infusion for about twenty-four hours, once in seven days, wherein the dose of the compound is about 160 mg/m² or greater.

23. The method of claim 22, wherein the dose of the compound is about 360 mg/m² or greater.

24. The method of claim 22, wherein the dose of the compound is about 720 mg/m² or greater.

25. The method of claim 22, wherein the dose of the compound is about 1370 mg/m² or greater.
